(19) United States
(12) Patent Application Publication
(10) Pub. No.: US 2022/0225897 A1
(43) Pub. Date: Jul. 21, 2022

(54) SYSTEMS AND METHODS FOR REMOTE MOTOR ASSESSMENT

(71) Applicant: Neurolutions, Inc., Santa Cruz, CA (US)

(72) Inventors: Kern Bhugra, Santa Cruz, CA (US); Eric Claude Leuthardt, St. Louis, MO (US)

(73) Assignee: Neurolutions, Inc., Santa Cruz, CA (US)

(21) Appl. No.: 17/648,384

(22) Filed: Jan. 19, 2022

Related U.S. Application Data
(60) Provisional application No. 63/199,729, filed on Jan. 20, 2021.

(51) Int. Cl. A61B 5/11; A61B 5/00; A61B 5/22

(52) U.S. Cl. CPC A61B 5/1128; A61B 5/6811; A61H 1/0288; A61B 5/225; A61B 2562/0219; A61B 5/743

(57) ABSTRACT

Systems for performing human motor assessments remotely include a wearable orthosis device having a sensor, an imaging device, and a computer. The computer includes instructions that cause the computer to perform a method comprising receiving images from the imaging device; making a measurement of a movement from the images, the movement performed by a patient; and calculating a motor assessment metric using the measurement of the movement and data from the sensor.

### RELATED APPLICATIONS

This application claims priority to U.S. Provisional Patent Application No. 63/199,729, filed on Jan. 20, 2021, and entitled “Systems and Methods for Remote Motor Assessment”; the contents of which are hereby incorporated by reference in full.

### BACKGROUND

Patients who have had a stroke can experience many types of damage after the event. Effects of a stroke can include issues related to movement of upper and lower extremities of the body such as impaired motor movement, paralysis, pain, weakness, and problems with balance and/or coordination. Rehabilitation programs for these motor impairments involve movement therapies to help the patient strengthen muscles and relearn how to perform motions.

To monitor a patient's sensorimotor recovery after a stroke, the Fugl-Meyer Assessment (FMA) is commonly used. The FMA is administered by a clinician such as a physical therapist or occupational therapist and involves assessing items in five domains—motor function, sensory function, balance, joint range of motion and joint pain. Different parts of the body are assessed for all of these different categories. For example, in a Fugl-Meyer Assessment for the Upper Extremity (FMA-UE), various motions and exercises for the shoulder, hand, wrist, elbow, forearm and fingers are performed in various positions. Some assessment items involve volitional movement while others involve passive motions. The measurements also involve assessing a patient's ability to grasp objects including a piece of paper, a pencil, a cylindrical object and a tennis ball. The scores for each item are totaled to result in an overall FMA score, where sub-scores of certain groups (e.g., upper arm, or wrist/hand) can also be evaluated. The FMA is repeated periodically to assess the patient's recovery over time.

Other types of motor assessments include the motricity index, action research arm test (ARAT), arm motor ability test (AMAT), and stroke impact scale (SIS). All of these assessments are important in helping to monitor and guide therapy for a patient as they undergo rehabilitation.

### SUMMARY

In some embodiments, systems for performing human motor assessments remotely include a wearable orthosis device having a sensor, an imaging device, and a computer. The computer includes instructions that cause the computer to perform a method comprising receiving images from the imaging device; making a measurement of a movement from the images, the movement performed by a patient; and calculating a motor assessment metric using the measurement of the movement and data from the sensor.

In some embodiments, systems for performing human motor assessments remotely include a wearable orthosis device, an imaging device, and a computer. The wearable orthosis device has a body part interface, a motor-actuated assembly coupled to the body part interface, and a sensor coupled to the body part interface. The computer includes instructions that cause the computer to perform a method comprising receiving images from the imaging device; making a measurement of a movement from the images, the movement performed by a patient; and calculating a motor assessment metric using the measurement of the movement and data from the sensor.

### BRIEF DESCRIPTION OF THE DRAWINGS

FIGS. 1A-1B are side views of an orthosis device for a hand, in accordance with some embodiments.

FIGS. 2A-2D are various views of an orthosis device for a hand, in accordance with some embodiments.

FIGS. 3A-3B show clinical and patient requirements for a remote Fugl-Meyer assessment, in accordance with some embodiments.

FIG. 4 is an isometric view of camera and user interface hardware for a remote motor assessment, in accordance with some embodiments.

FIGS. 5A-5B are images of skeletal tracking of arm movement during a remote motor assessment, in accordance with some embodiments.

FIGS. 6A-6F are images of skeletal tracking of hand and finger movement during a remote motor assessment, in accordance with some embodiments.

FIG. 7 is a block diagram of methods for performing remote motor assessments, in accordance with some embodiments.

FIG. 8 is a diagram of an example hand movement assessment, in accordance with some embodiments.

### DETAILED DESCRIPTION

A substantial unmet clinical need and large commercial opportunity exists in chronic stroke patients' poor access to advanced rehabilitation care. Although many approaches have been developed for post-stroke motor therapy, approximately 65% of stroke patients with hemiparesis remain unable to use their affected hand six months after their stroke. When considering rehabilitation, having objective metrics to assess a patient's motor function is critical to define the nature of a deficit and the impact of treatment. One of the most widely recognized evaluations is the Fugl-Meyer assessment of sensorimotor function, which requires an in-person interaction with an occupational therapist (OT). Other commonly used motor assessments such as the motricity index, ARAT, and AMAT also involve in-person interaction.

Telehealth is the use of any communication modality (e.g., integrated video and audio, video teleconferencing, etc.) that enables physical separation of patient and practitioner while delivering health care services at a distance. Telerehabilitation (TR) uses telehealth technologies to provide distant support, assessment and information to people who have physical and/or neurological/cognitive impairments. Conventional assessment tools such as the Fugl-Meyer assessment are insufficient to fully enable a home-based telehealth capability. The in-person requirement of the FMA is inefficient and tethers the patient to an institutional visit to perform. The manpower requirements unnecessarily act as a bottleneck for quantifying the impact of a telehealth rehabilitation intervention.

The present disclosure describes a home-based system for performing physical motor assessments that are equivalent to conventional in-person methods. Although embodiments shall primarily be described in relation to the Fugl-Meyer assessment, the present methods and systems also apply to other types of motor assessments such as the motricity index, ARAT and AMAT.

Embodiments utilize a brain-computer interface (BCI) based orthosis device in combination with a sensor system and associated computer vision techniques. Embodiments of the systems and methods enable nearly identical physical interactions as an in-person motor assessment and produces comparable functional metrics. This telehealth evaluation capability shall be referred to as a Remote Automatic Evaluation (RAE), or “RAE-FM” when referring to a remote Fugl-Meyer assessment. This novel remote assessment capability can enable scalable, cost-efficient assessments of patients in their home environments. This technology is significant not only for understanding the impact of BCI rehabilitation therapy, but for understanding other remotely delivered rehabilitation interventions.

The remote assessments disclosed herein provide further benefits in addition to the home-based capability. In some embodiments, the remote assessment system involving the BCI-based orthosis device can simplify the assessment routines and/or metrics being measured. In other embodiments, the remote assessment system can provide information beyond that which can be assessed by conventional methods. For example, use of the orthosis device can provide quantifiable degrees of measurement rather than qualitative assessments such as “none/partial/full” scoring categories as used in the conventional FMA. In another example, the remote assessments can provide metrics that are not possible without the orthosis device, such as measuring a level of assistance needed from the orthosis device to perform a task or measuring a gripping force the patient exerts on an object. In further embodiments, the remote assessments can provide customized therapy plans based on the individual patient's progress. As shall be described in this disclosure, the present systems and methods enable human motor assessments to be performed remotely, while enhancing the efficiency and quality of the assessments.

Telehealth BCI-rehabilitation has been evolving in the stroke rehabilitation field as a powerful approach for providing improved outcomes and health care access for chronic stroke patients. In the setting of chronic stroke, most therapeutic techniques are generally ineffective due to the severe reduction in ability to achieve recovery beyond three months post-stroke. Some promising techniques such as constraint-induced movement therapy (CIMT) have been successfully used to combat “learned non-use” of affected limbs. These techniques, however, are not scalable to a broad stroke population due to many patients lacking the necessary level of motor function.

The use of brain-computer interfaces is an emerging technology for post-stroke motor rehabilitation in the chronic setting. BCI technology involves the acquisition and interpretation of brain signals to determine intentions of the person that produced the brain signals and using the determined intentions to carry out intended tasks. BCI technology has been explored in connection with the rehabilitation of impaired body parts, including rehabilitation of upper extremity body parts such as arm and hand function impaired due to a stroke event. BCI-mediated stroke therapy allows patients who have motor impairments too severe for traditional therapy to still achieve a functional recovery. BCIs may also be effective for promoting recovery through plasticity by linking disrupted motor signals to intact sensory inputs.

Generally, BCIs do not require patients to generate physical motor outputs. There is a strong premise that BCI-based approaches can be used to develop treatments for patients who are unable to achieve recovery through more traditional methods. Several recent BCI-based treatments in the industry have been shown to aid in motor recovery in chronic stroke patients through varied approaches, such as electrical stimulation or assistive robotic orthoses. BCI-based approaches are thought to drive activity-dependent plasticity by training patients to associate self-generated patterns of brain activity with a desired motor output. Classically, changes in the distribution and organization of neural activity have been identified as a potentially important factor in achieving motor recovery. Motor control is thought to shift to perilesional regions when the primary motor cortex is damaged. Local neural reorganization, however, may not be sufficient for recovery if cortical damage is too severe, or if the ipsilesional corticospinal tract (CST) is substantially transected. Since rehabilitative BCIs often use perilesional or ipsilesional signals, they may not be as effective as rehabilitation systems for patients experiencing high levels of motor impairment. Studies performed in relation to this disclosure have shown that signals acquired from electroencephalogram (EEG) electrodes placed over healthy contralesional motor cortex can be used for BCI control, and that the use of such a system can induce robust functional improvements in chronic stroke patients.

While non-invasive BCI-rehabilitation technologies are promising for recovering motor function in chronic stroke patients, for these approaches to fully achieve their potential they must be made more accessible and scalable. Telehealth approaches have the ability to overcome the current accessibility barrier. Telehealth rehabilitation has received significant attention in recent years. Prior to the COVID-19 pandemic, telehealth delivery of care has been a growing trend; and since the onset of the COVID-19 pandemic, telehealth has become an essential tool for continuing to deliver health care to patients. This is reflected by changing regulations and reimbursement policies at the federal and state level to support expansion of the application of telemedicine. Even after the COVID-19 pandemic has passed, these changes are likely to remain.

Effective implementation of a telehealth rehabilitation (TR) program has been shown to increase access to service and result in improved rehabilitation outcomes for individuals with physical impairments after discharge to home. First, in addition to pandemic situations, TR can benefit people with disabilities residing in rural, remote locations because these individuals face incomplete service networks that threaten their safety and independent functioning. Rural individuals also face more barriers in accessing care because they travel farther to medical and rehabilitation appointments and have more transportation problems than their urban counterparts. This situation is especially exasperated for severely affected stroke patients whose mobility is even more severely impaired. Indeed, the farther rehabilitation programs are from residents' homes, the less likely residents are to receive services. Second, TR can be provided at less cost than in-person services and can eliminate patients' travel time between their homes and the rehabilitation clinic. Third, TR reduces the need for therapists/technologists to travel to patients' homes while supporting real-time interactions with patients with physical disabilities in their home settings. Fourth, an effective TR program can enhance continuity of care by enabling communication with the caregivers. Finally, there does not seem to be a tradeoff between remote versus in-clinic regarding outcomes. Activity-based training produced substantial gains in arm motor function regardless of whether it was provided via home-based telerehabilitation or traditional in-clinic rehabilitation.

The present disclosure describes methods and systems for providing in-home assessment of motor function after stroke, which can greatly increase the number of stroke patients that can be evaluated and ultimately treated. Furthermore, the ability for patients to perform assessments at home enables the patient's rehabilitation program to be tailored more specifically to their individual needs and progress, thus improving their overall recovery. There is a significant need to create a remote, telehealth approach that can evaluate and ultimately treat chronic stroke patients in the safety of their own home.

Methods and systems of this disclosure utilize an electro-mechanical orthosis device in conjunction with sensors and a camera system that enable physical interactions nearly identical to those used in conventional assessments such as an FMA and produces comparable functional metrics. The orthosis device can be used to achieve at least a portion of the measurements in a motor assessment testing routine. Specially designed algorithms and software with machine learning are also described that provide the ability to track and evaluate an individual's motions for a remote motor assessment and personalize the evaluation for the specific patient's needs. Embodiments shall be described primarily in terms of upper extremity assessments, particularly the hand. However, embodiments also apply to other parts of the body such as the lower extremity.

Orthoses in the rehabilitation industry have used various mechanisms to accomplish movement and/or assistance in the movement of impaired body parts. One such mechanism is to physically attach or secure an active movable portion of the orthosis device to the body part that is to be moved or for which movement is to be assisted. The active movable portion of the orthosis device secured to the body part may then be activated to move by a motor or some other form of actuation, and as such accomplish or assist in the movement of the impaired body part secured thereto. Another such mechanism to accomplish or assist in the movement of a body part is through a technique called functional electrical stimulation (“FES”), which involves the application of mild electrical stimuli to muscles that help the muscles move or move better.

Examples of BCI-based systems for use with impaired body parts include descriptions in U.S. Pat. No. 9,730,816 to Leuthardt et al. (816 patent), under license to the assignee of the present patent application, the contents of which is incorporated by reference herein. The '816 patent describes the use of BCI techniques to assist a hemiparetic subject, or in other words, a subject who has suffered a unilateral stroke brain insult and thus has an injury in, or mainly in, one hemisphere of the brain. For that patient, the other hemisphere of the brain may be normal. The '816 patent describes an idea of ipsilateral control, in which brain signals from one side of the brain are adapted to be used, through a BCI training process, to control body functions on the same side of the body.

Additional examples of BCI-based systems for use with impaired body parts include descriptions in U.S. Pat. No. 9,539,118 to Leuthardt et al. (118 patent), which is commonly assigned with the present patent application and incorporated herein by reference. The '118 patent describes wearable orthosis device designs that operate to move or assist in the movement of impaired body parts, for example body parts that are impaired due to a stroke event, among other conditions described in the '118 patent. For example, the '118 patent describes rehabilitation approaches for impaired fingers, among other body parts including upper as well as lower extremities, using wearable orthosis devices that operate to move or assist in the movement of the impaired body part and that are controlled using BCI techniques. The '118 patent further elaborates BCI-based rehabilitation techniques that utilize brain plasticity to “rewire” the brain to achieve motor control of impaired body parts.

Further examples of BCI-based systems for use with impaired body parts include descriptions in U.S. patent application Ser. No. 17/068,426 ('426 application), which is commonly assigned with the present patent application and which is incorporated herein by reference. The '426 application describes wearable orthosis device designs that operate to move or assist in the movement of impaired body parts, such as those impaired due to a stroke event, among other conditions described in the '426 application. For example, the '426 application describes an orthosis system that can be operated in one or more of: (i) a BCI mode to move or assist in the movement of the impaired body part based on an intention of the subject determined from an analysis of the brain signals, (ii) a continuous passive mode in which the orthosis system operates to move the impaired body part, and (iii) a volitional mode in which the orthosis system first allows the subject to move or attempt to move the impaired body part in a predefined motion and then operates to move or assist in the predefined motion, such as if the system detects that the impaired body part has not completed the predefined motion.

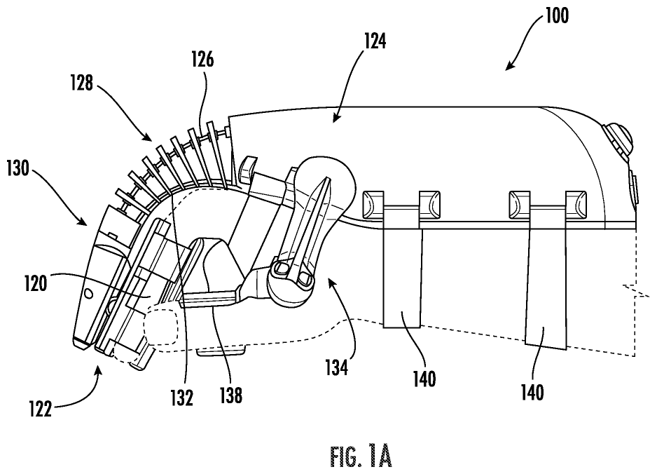

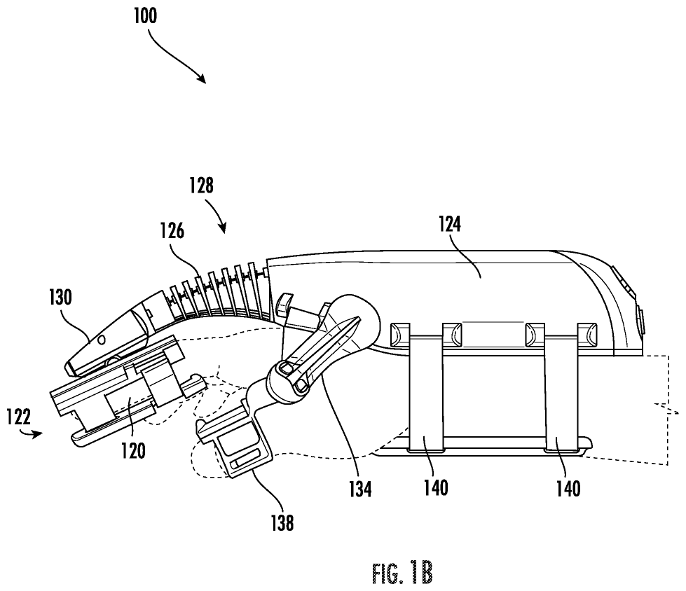

An embodiment of an orthosis device of the '426 application is shown in FIGS. 1A-1B. The orthosis device 100 is shown in a flexed or closed position in FIG. 1A, and in an extended position in FIG. 1B. The wearable orthosis device 100 may receive transmitted signals (for example, wirelessly) containing information about the brain signals acquired by a brain signal acquisition system (e.g., an EEG-based or electrocorticography-based electrodes headset). The orthosis device 100 may then process those received signals to determine intentions using embedded processing equipment, and in accordance with certain detected patient intentions cause or assist the movement of the patient's hand and/or fingers by robotic or motor-drive actuation of the orthosis device 100.

Orthosis device 100 includes a main housing assembly 124 configured to be worn on an upper extremity of the subject. The main housing assembly 124 accommodates straps 140 to removably secure the main housing assembly 124 and thus the other attached components of the orthosis device 100 to the forearm and top of the hand. The straps 140 may be, for example, hook-and-loop type straps. The main housing assembly 124 comprises a motor mechanism configured to actuate movement of a body part of the upper extremity of the subject. A flexible intermediate structure 128 is configured to flex or extend responsive to actuation by the motor mechanism to cause the orthosis device 100 to flex or extend the secured body part. The wearable orthosis device 100 is designed and adapted to assist in the movement of the patient's fingers, specifically the index finger 120 and the adjacent middle finger (not visible in this view), both of which are securely attached to the orthosis device 100 by a finger stay component 122. The patient's thumb is inserted into thumb stay assembly 134 which includes thumb interface component 138. The main housing structure 124 is designed and configured to be worn on top of, and against, an upper surface (that is, the dorsal side) of the patient's forearm and hand. The finger stay component 122 and thumb stay assembly 134 are body part interfaces secured to the body part (finger or thumb, respectively). A motor-actuated assembly connected to the body part interface moves the body part interface to cause flexion or extension movement of the body part.

The motor-actuated assembly may be configured as a linear motor device inside the main housing structure 124. The linear motor device longitudinally advances and retracts a pushing-and-pulling wire 126 that extends distally from the distal end of the main housing structure 124 and extends longitudinally through the flexible intermediate structure 128 and connects to a connection point on a force sensing module (“FSM”) 130. The flexible intermediate structure 128 has a flexible baffle structure. When a linear motor in the main housing structure pulls the wire 126 proximally, the attached FSM 130 is pulled proximally. This motion causes the flexible intermediate structure 128 to flex so its distal end is directed more upwardly, causing or assisting in extension movement of the secured index and adjacent middle fingers. The upward flexing of the flexible intermediate structure 128 so that its distal end is directed more upwardly (and also its return) is enabled by the baffle structure of the flexible intermediate structure 128. In particular, a generally flat bottom structure 132 is provided on the flexible intermediate structure 128, where the bottom structure 132 is configured to attach to a bottom or hand-side of each of the individual baffle members. The opposite or top-side of each of the individual baffle members are not so constrained and thus are free to be compressed closer together or expanded further apart by operation of the pushing-and-pulling wire 126 enlarging and/or reducing the top-side distance between the distal end of the main housing structure 124 and the proximal end of the FSM 130.

The FSM 130 serves a force sensing purpose, comprising force sensors that are capable of measuring forces caused by patient-induced finger flexion and extension vis-à-vis motor activated movements of the orthosis device 100. The force sensing function of the FSM 130 may be used, for example, to ascertain the degree of flexion and extension ability the patient has without assistance from the orthosis device 100, to determine the degree of motor-activated assistance needed or desired to cause flexion and extension of the fingers during an exercise, or other purposes.

In embodiments of this disclosure, electro-mechanical orthosis devices are used to acquire significant amounts of meaningful data about a patient's clinical performance, including monitoring utilization data, open/close success rates, force profile characteristics, accelerometer info as well as motor position metrics related to range of motion. For example, force sensors in the FSM 130 can measure passive hand opening force (spasticity), active grip strength and extension force. Housing 124 may contain a six-axis inertial measurement unit (IMU) that has an accelerometer and gyroscope to monitor motion sensing, orientation, gestures, free-fall, and activity/inactivity. A motor potentiometer to measure position for facilitating the evaluation of the range of motion may also be included in either the FSM 130 or housing 124. The orthosis device 100 has substantial sensor and mechanical capabilities to physically interact with the limb and hand of a stroke patient which can be leveraged to provide remote functional metrics comparable to portions of those performed in a conventional in-person motor assessment such as a Fugl-Meyer evaluation.

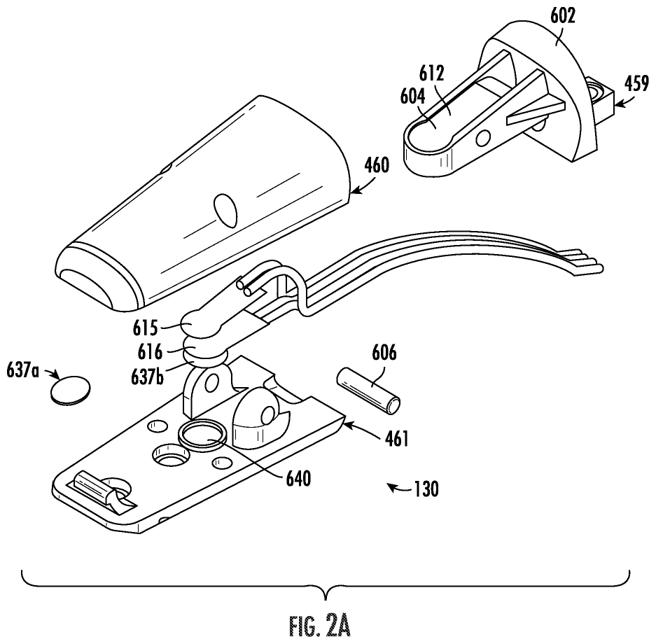

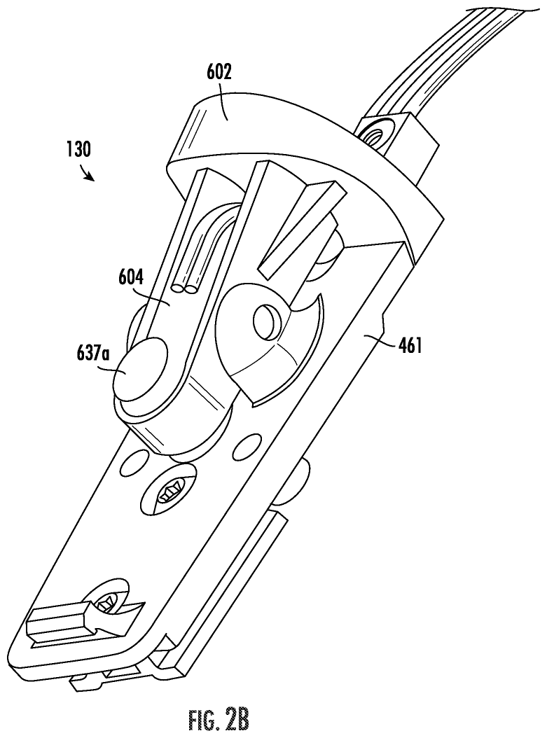

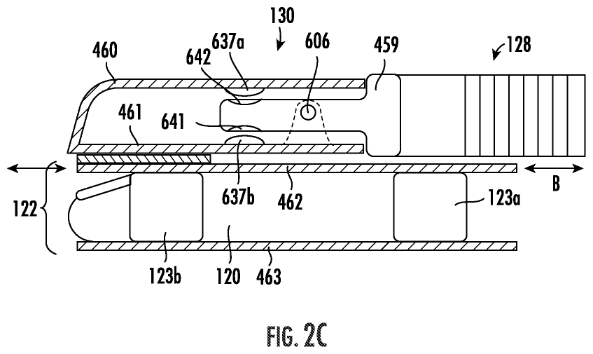

FIGS. 2A-2C show details of sensors in the FSM 130 of orthosis device 100, in accordance with some embodiments. Further description of these sensors may be found in the '426 application, incorporated by reference above. In FIGS. 2A-2B, a vertically oriented proximal end plate 602 has an elongate extension component 604 extending distally from the end plate 602. The elongate extension component 604 serves as a carrier of two force sensing resistors 615, 616. A horizontally oriented dividing wall 612 of the extension component 604 separates the structure of the two force sensing resistors (“FSRs”) 615, 616 from one another, or in other words, separates a first FSR 615 that may be assembled to be located above the dividing wall 612 (hereafter called the “top” FSR 615) from a second FSR 616 that may be assembled to be located below the dividing wall 612 (hereafter called the “bottom” FSR 616).

To provide the force sensing capability of the connecting/FSM assembly 130, two force sense resistor (“FSR”) bumpers, buttons, or plungers 637a, 637b are utilized. A first FSR bumper 637a is fixedly positioned on an underside surface of an upper shell 460 of FSM 130 in a location thereon aligned with the top FSR 615, so that the top FSR's upwardly facing surface (that is, its force sensing surface, labeled as 642 in FIG. 2C) comes in contact with and bears upon the first FSR bumper 637a when a distal end of the central support 459 rocks or pivots upwardly relative to the fixed-together upper and lower shells 460, 461. A second FSR bumper 637b is fixedly positioned onto and within an opening or recess 640 provided on a top surface of the lower shell 461 in a location thereon aligned with the bottom FSR 616, so that the bottom FSR's downwardly facing surface (that is, its force sensing surface, labeled as 641 in FIG. 2C) comes in contact with and bears upon the second FSR bumper 637b when a distal end of the central support 459 rocks or pivots downwardly relative to the fixed-together upper and lower shells 460, 461.

When the distal end of the central support 459 rocks or pivots downwardly relative to the upper and lower shells 460, 461, the force sensing surface 642 of first FSR 615 may become no longer in contact with the first bumper 637a; and when the distal end of the central support 459 rocks or pivots upwardly relative to the upper and lower shells 460, 461, the force sensing surface 641 of second FSR 616 may become no longer in contact with the second bumper 637b. The rocking or pivoting of the central support 459 may be limited by constraints imposed by the clearances of the two bumpers 637a, 637b from their respective FSRs 615, 616. In some embodiments, such clearances are minimized so that the amount of rocking or pivoting permitted is minimized but the force-sensing functioning of both FSRs is still enabled.

FIG. 2C is a side cross-sectional view illustrating that finger stay component 122 is slidably engaged with the underside of the lower shell 461. This sliding engagement is illustrated by arrow B. Accordingly, the lower shell 461 of the connecting/FSM assembly 130 is connected to the finger stay component 122 that is attached thereunder in a manner that the angular orientation of the FSM 130 and the finger stay component 122 remain fixed, and yet the finger stay component 122 is permitted to freely move or slide longitudinally with respect to the lower shell 461. The finger stay component 122 is provided with an upper plate 462 that rests above the two secured fingers 120 and a lower generally horizontal plate 463 that rests below the two fingers. Two adjustable straps 123a, 123b are provided with the two plates 462, 463 to secure the index and middle fingers as a unit between the two plates.

A discussion of how these force sensing capabilities may be utilized in an orthosis device shall be described in reference to FIG. 2C. In a first example, the orthosis device is not being actuated but that the patient is opening/extending his or her fingers under his or her own force. The orthosis device is able to be “forced” open (that is, forced into an “extended” position) by the patient's own finger opening force, which in some cases may involve activating a motor associated with orthosis device to be enabled to “follow” the volitional action of the subject. In other words, although it is the patient's own finger operating force that induces such movement in the orthosis device, the linear actuator may be “turned on” to allow the fingers to open with the patient's own force (without assist). The patient's own finger opening force causes a portion of the lower shell 461 distal of the pivot point/dowel 606, including the bottom bumper 637b affixed thereto, to be moved upwardly relative to portion of the central support that is also distal of the pivot point/dowel 606, such that the dome surface of the bottom bumper 637b contacts and applies a force against the downward facing sensing surface 641 of the bottom FSR 616. As such, the bottom FSR 616 captures a measurement from which the patient's finger opening force may be determined.

In a second scenario, the patient is closing/flexing his or her fingers under his or her own volition and the orthosis device again is not being actuated but is able to “follow” the subject's volitional action so that the orthosis device may be “forced” into a flexed or closed position by the patient's own finger closing force. In this second scenario, the patient's own finger closing force causes a portion of the upper shell 460 that is distal of the pivot point/dowel 606, and thus the top bumper 637a affixed thereto, to be “pulled” downwardly such that the domed surface of the top bumper 637a is put in contact with and applies a force against the upwardly facing sensing surface 642 of the top FSR 615. As such, the top FSR 615 enables measurement of a patient's “finger closing force.”

In another scenario, the orthosis device is actuated to open/extend the finger stay component 122 and hence open/extend the patient's fingers secured thereto, but the patient is not able to provide any finger opening/extension force. In this case, the flexible intermediate structure 128 may be actuated so that its distal end is oriented more upwardly to move the connecting/FSM assembly's central support 459 upwardly and in a clockwise direction. Because in this scenario it is assumed that the patient will be providing no help in opening the fingers, a distal portion of the upper and lower shells 460, 461 will “rock” downwardly in a counter-clockwise direction relative to the central support 459 so that the upwardly facing sensing surface 642 of the top FSR 615 comes in contact with and bears against the top bumper 637a affixed to the inner surface of the upper shell 460. In this case, the downwardly facing sensing surface 641 of the bottom FSR 616 will no longer be in contact with the bottom bumper 637b affixed to the lower shell 461. In this scenario, the presence of a force at the top FSR 615 and absence of a force at the bottom FSR 616 may thereby inform the orthosis device that the patient is providing little or no assistance in the finger opening/extension movement that is being actuated by the orthosis device.

In an opposite scenario, the orthosis device is actuated again, this time to close or flex the finger stay component 122 and hence close or flex the patient's fingers. In this scenario, the patient is not able to provide any finger closing or flexing force, but instead will be moved into a flexed position by operation of the orthosis device. In this case, the flexible intermediate structure 128 is actuated so that its distal end becomes oriented more downwardly, which in turn causes the connecting/FSM assembly's central support 459 to be moved downwardly in a counter-clockwise direction. Because in this scenario the patient is providing no help in closing the fingers, the fixed-together upper and lower shells 460, 461—which again are in a fixed angular orientation with respect to the finger stay component 122 and hence to the patient's fingers—will then “rock” in a clockwise direction relative to the central support 459 until the downwardly facing sensing surface 641 of the bottom FSR 616 comes into contact with and bears against the bottom bumper 637b affixed to the lower shell 461. In addition, the upwardly facing sensing surface 642 of the top FSR 615 will then be free of contact with the top bumper 637a affixed to the upper shell 460. In this scenario, the presence of a force at the bottom FSR 616 and absence of a force at the top FSR 615 may thereby inform the orthosis device that the patient is not providing any assistance in the finger closing/flexing movement that is being actuated by the orthosis device.

In yet another scenario, the orthosis device is actuated to open/extend the finger stay component 122, but the patient is providing a full finger opening force beyond the opening/extension force being provided by the orthosis device. In this scenario, despite the fact that the flexible intermediate structure 128 is providing a force that would move the central support 459 upwardly, the patient is providing an additional opening/extending force on the finger stay component 122 and thus on the upper and lower shells 460, 461 angularly affixed thereto, and as such, the patient is volitionally causing the upper and lower shells 460, 461 to move at even faster rate than the actuated central support 459 is being actuated by the orthosis device. As such in this scenario, the bottom bumper 637b affixed to the lower shell 461 may come in contact with and bear against the bottom FSR's downward facing sensing surface 641, and the top bumper 637a affixed to the upper shell 460 may then be free of and thus provide no force against the top FSR's upward facing sensing surface 642. As such, in this scenario the presence of a force sensed at the bottom FSR 616, and absence of a force sensed at the top FSR 615 may inform the orthosis device that the patient is providing all of the necessary finger opening force to achieve the desired finger opening/extending.

In other implementations, load cell force sensing may be used in connection with the pushing-and-pulling wire 126 (FIGS. 1A-1B), to provide for the above-described force sensing capabilities. In one implementation shown in FIG. 2D, a sensor 234 and a coupler 236 are mounted on an end of a motor mechanism 230 that includes a motor and a linear actuator. The sensor 234 may be, for example, a load cell with wiring 235. Other types of sensors may be used such as position sensors (e.g., optical, proximity), other force sensors (e.g., strain gauge, pressure sensor), and limit switches. The coupler 236 receives and holds the pushing-and-pulling wire 126. The load cell force sensor 234 is in the form of a cylindrical drum-shaped structure may be provided in series with the pushing-and-pulling wire 126, for example, with one side of the drum-shaped structure facing proximally and the opposite side of the drum-shaped structure facing distally. In this implementation, the pushing-and-pulling wire 126 may comprise two portions of wire, a proximal portion of wire 126 and a distal portion of wire 126. The proximal portion of the pushing-and-pulling wire 126 may have its proximal end attached to a distal end of a linear motor inside the main housing structure 124, and its distal end fixedly attached to a proximally facing side of the load cell drum shaped structure. The distal portion of the pushing-and-pulling wire 126 may have its proximal end fixedly attached to a distally facing side of the load cell drum-shaped structure and its distal end fixedly attached to the force sensing module 130 (FIGS. 2A-2C).

A load cell force sensor design may be selected that is capable of sensing both a tension force (exerted on the load cell force sensor, for example, by a pushing-and-pulling wire 126 being extended distally against the load cell force sensor) and a compression force (exerted on the load cell force sensor, for example, by a pushing-and-pulling wire being pulled proximally to effectively “pull” on the load cell force sensor). Accordingly, such an implementation of a force sensing module may provide functionality in connection with a volitional mode of operation of the orthosis device.

Other types of sensors may be included in orthosis devices that are used for the remote motor assessments of the present disclosure. For example, accelerometers, gyroscopes, and/or potentiometers may be used to measure position, speed, acceleration, and/or orientation. Furthermore, any of the sensors described in this disclosure may be used in orthosis devices of types other than that shown in FIGS. 1A-1B and 2A-2D. For example, embodiments may utilize orthosis devices for other movements of the upper extremity such as the elbow or shoulder. In other examples, embodiments may utilize orthosis devices for the lower extremity, where sensors may be used to detect forces and movement of the hip, knee, ankle, foot, and toes.

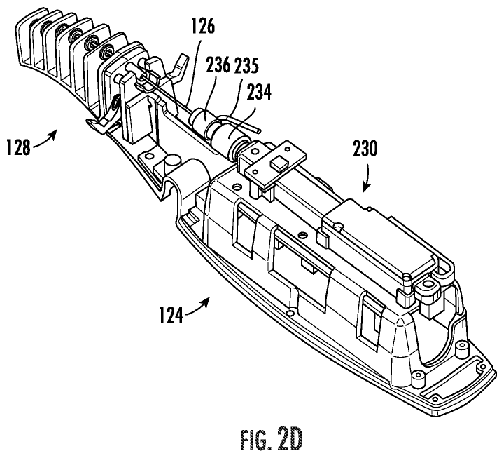

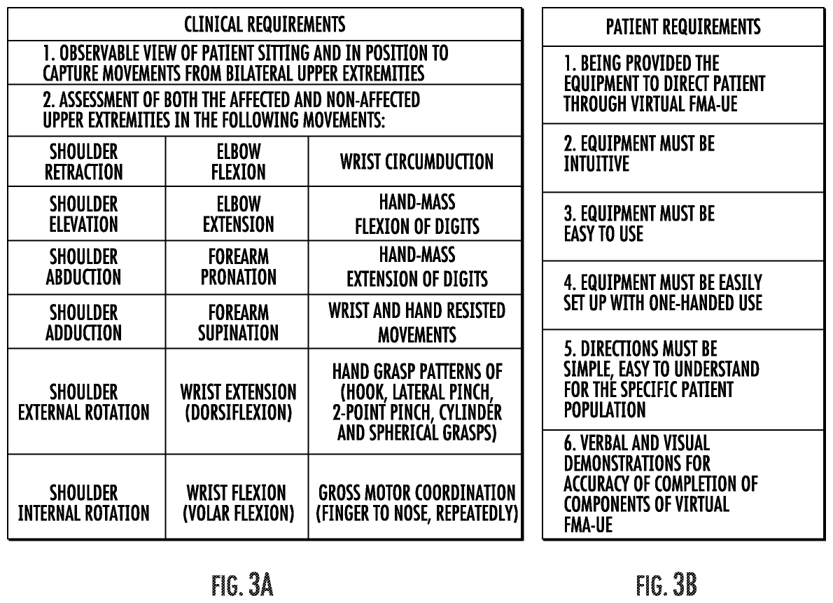

The features required to capture a virtual motor assessment, such as a Fugl-Meyer or other motor assessment, must address and satisfy end user requirements. For the virtual assessments of the present disclosure, there are two end users—the clinician and the patient—each with a different set of requirements to be addressed. Examples of clinical criteria for the upper extremity are listed in FIG. 3A, while examples of patient criteria are listed in FIG. 3B. The clinical criteria reflect the various body parts (shoulder, elbow, forearm, wrist, hand) and motions of each body part may undergo for an upper body evaluation (e.g., the FMA-UE). The patient requirements of FIG. 3B illustrate that a remote assessment must be easy to use and follow, guiding a patient through the various steps so that the evaluations can be performed accurately. Although the requirements shown in FIGS. 3A-3B are for the upper extremity, embodiments may similarly apply to other parts of the body such as a Fugl-Meyer Assessment of the Lower Extremity (FMA-LE), where motions of the hip, knee, ankle and foot are assessed. Some or all of the criteria in FIGS. 3A-3B can be included in the systems and methods of the present disclosure. In further embodiments, other criteria and movement assessments may be added besides those shown in FIGS. 3A-3B. In embodiments, the motor assessment metric being assessed by the system is a metric in a Fugl-Meyer assessment, a motricity index, an action research arm test (ARAT), an arm motor ability test (AMAT), or a stroke impact scale (SIS).

Using the Fugl-Meyer Assessment of the Upper Extremity as an example, the conventional FMA-UE has four sections, each having specific subtests to complete. These specific sections look at active movement of the upper extremity, forearm, wrist, hand and coordination/speed. In these subtests the scoring generally is noted as: 0—No active movement, 1—Partial active movement, 2—Full range of active movement. The coordination/speed subtests include qualitative evaluations of tremor, dysmetria, and time to do a movement. The FMA-UE also includes grasping motions such as a hook grasp, thumb adduction, pincer grasp, cylinder grasp, and spherical grasp. The grasping motions are rated in three categories: cannot be performed, can hold position/object but not against a tug, and can hold position/object against a tug. Traditionally the FMA-UE is administered by the clinician who visually observes the movements for scoring.

The AMAT uses components such as mugs, combs, and jars, and requires the patient to perform tasks or movements which may be further divided into subtasks. The tasks/subtasks are timed and evaluated on the ability to perform the task and how well the task is performed.

The ARAT has four subtests of grasp, grip, pinch, and gross movement, and utilizes tools such as wood blocks, balls, and drinking glasses. The tasks are evaluated on a four-point scale: 0—no movement, 1—partially performed, 2—completed but takes abnormally long, 3—performed normally. Example movements include grasping blocks of wood of different sizes, pouring water from glass to glass, holding a ball bearing with their finger and thumb (pinching), and placing their hand on their head.

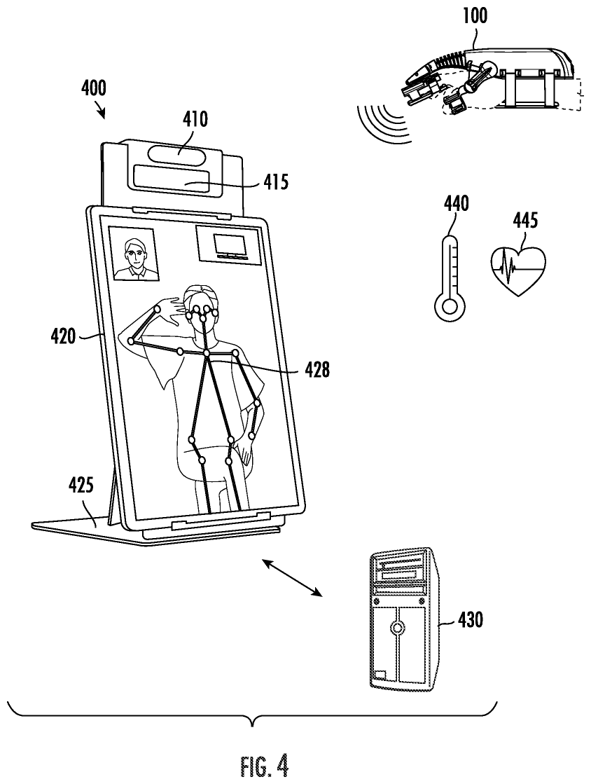

The systems and methods of the present disclosure utilize imaging devices along with the orthosis device sensors to remotely measure movements performed in motor assessments. FIG. 4 illustrates an example system 400 that includes a depth camera 410, a tracking camera 415, and a computing device 420 on an optional stand 425. In FIG. 4 the computing device 420 is illustrated as a tablet computer. In other embodiments, the computing device 420 can be, for example, a mobile phone, laptop computer or a personal computer. The system may be configured to communicate with the mobile device, and the mobile device displays directions for performing the movement. The computing device 420 communicates with the orthosis device 100 and may also be connected to a central processor 430 such as a cloud computing system.

The patient's care team, which may be the patient's physician, medical care team or other third party, can remotely access results of the patient's motor assessments through the computing device 420 and/or central processor 430. The care team can then provide input and recommendations on the patient's ongoing therapy plan and/or future motor assessments. In some embodiments, the patient's physician or therapist can view the assessment session remotely (i.e., in a location different from the patient) while the patient is performing the motor assessment tests. In some embodiments, the patient performs the assessment session on their own and the physician or therapist views the results after the testing has been completed.

The computing device 420 displays a custom user interface 428 with guided motor assessment instructions, such as for directing a patient on movements for subtasks in a FMA-UE. Additional information from sensors such as environmental sensors 440 (e.g., for room temperature) and biological sensors 445 (e.g., for heart rate) may also be supplied to the computing device 420 and central processor 430 for use in the analysis of the motor assessments.

Embodiments leverage commercial camera hardware (e.g., depth camera 410 and tracking camera 415) and customized skeleton tracking software stored in computing device 420 to extend capabilities in remote assessment of motor function (e.g., upper or lower extremity function) in addition to those that can be obtained from orthosis devices (e.g., device 100). Camera hardware may be stereoscopic or non-stereoscopic. Example technology that may be utilized for the application and implementation of the virtual RAE-FM may include, but are not limited to, INTEL® RealSense Skeleton Tracking SDK with Depth Mapping (by Cubemos), Intel RealSense Hand Tracking, IpsiHand System tablet computer from Neurolutions (Santa Cruz, Calif.), Neurolutions Integrated Tablet Stand, and Neurolutions IpsiHand orthosis device (e.g., device 100).

In an example embodiment, the Intel RealSense Skeleton Tracking SDK with Depth Mapping employs cameras that use active infrared stereo vision to provide accurate depth and position of objects within its field of view, while the SDKs use this data to build a skeleton model with 18 joints within the body frame in 2D/3D. This camera (or similar cameras) allows the measurement and comparison of specific joint angles and upper/lower extremity rotation when completing specific and active movements of the upper/lower extremities. Conventionally, these measurements and comparisons are completed by visual observation of the assessor. Implementation of skeleton tracking with depth mapping allows the addition of precision and accuracy to visual observation assessment by the clinician. For example, the Intel RealSense Hand Tracking allows the joints and bones of the hands to be tracked using 22 points of tracking. Motion tracking software used in embodiments of the present disclosure can assess hand movements as directed when completing motor assessments such as the RAE-FM. The tablet stand is designed to position the cameras and the user interface—such as the 15″ IpsiHand touchscreen tablet personal computer (PC) and the depth cameras (e.g., Intel D435 or D455) and tracking cameras (e.g., Intel T265)—at a comfortable angle for seated assessment. The cameras may connect to the tablet PC via, for example, USB ports.

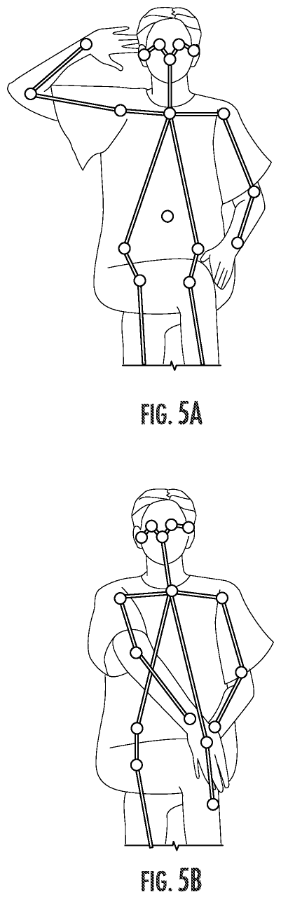

Demonstrated in FIGS. 5A-5B are images of example joint angles and movement captured while a subject is performing Section A Part 2 (Volitional Movement with Synergies) of the FMA-UE. The movements of Section A Part 2 require extensor synergy by having the patient move the affected limb from the ipsilateral ear (FIG. 5A) to the contralateral knee (FIG. 5B). Embodiments of the present disclosure use 3D cameras and tracking software to quantify the movements performed during a motor assessment.

The images of FIGS. 5A-5B were captured with Intel®'s Realsense™ D435 depth cameras with the Cubemos full body skeleton tracking artificial intelligence (AI) software development kit (SDK). The cameras use active infrared stereo vision to capture accurate depth and position of objects within its field of view, while the SDK uses the resulting 2-dimensional frame and the depth frame to build a skeleton model with 18 joints superimposed on the body. Joint coordinates are updated at, for example, >10 Hz and output to a custom algorithm that determines the real-time angles of each connecting line followed by a machine learning model that rates overall quality of skeletal motion (e.g., efficiency and stability of the movement) against the motor assessment (e.g., Fugl-Meyer) baseline. The skeleton tracking enables quantifiable precision of the movements and increased accuracy in correctly scoring of each movement. These findings demonstrate the ability of the present systems and methods to use camera and computer vision technology along with specially designed algorithms and machine learning to complement orthoses devices in creating a remote assessment tool.

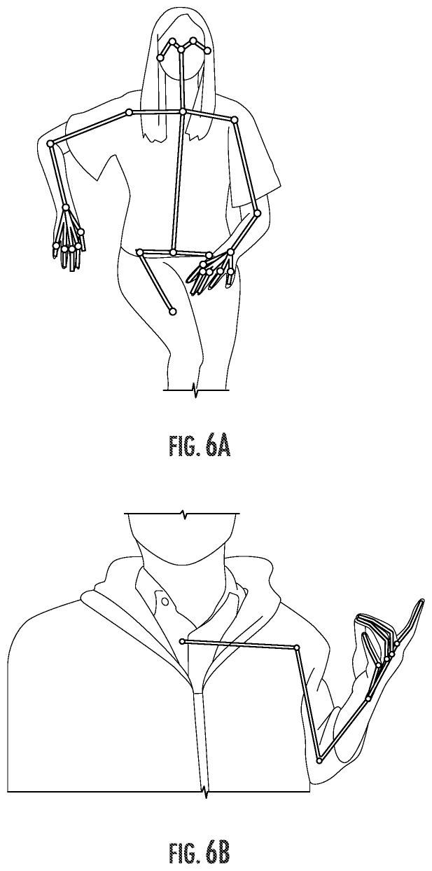

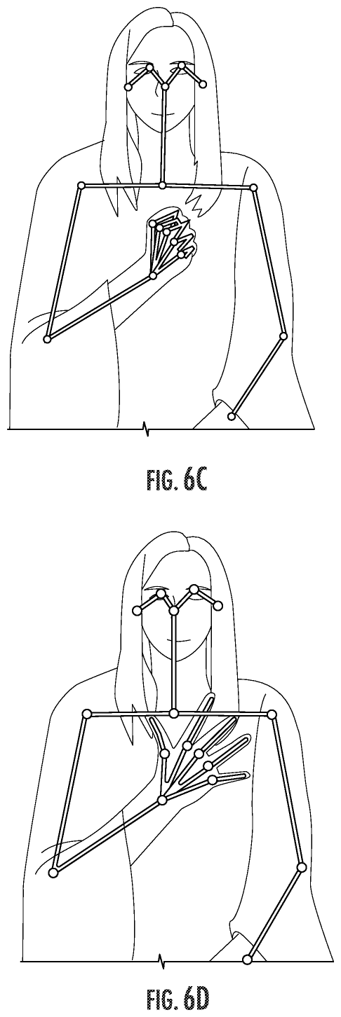

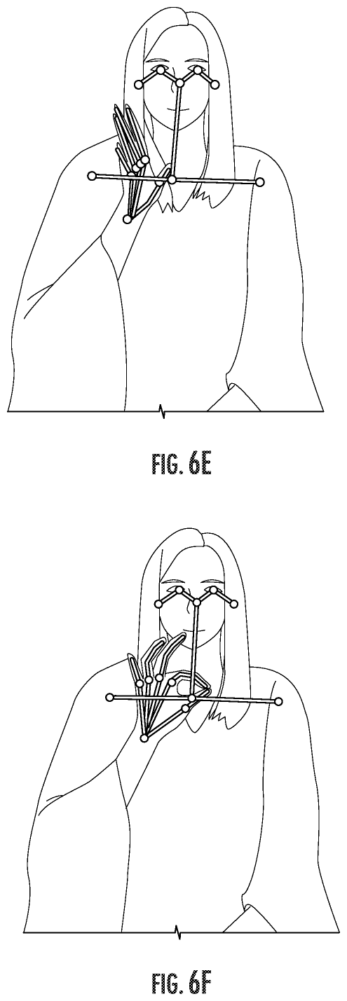

Tracking of hand and finger movements may also be performed in accordance with some embodiments, such as shown in FIGS. 6A-6F. FIGS. 6A and 6B are images showing recognition of individual fingers and their joints by the skeleton tracking software, in addition to the overall torso and arm and leg joints. FIGS. 6C and 6D are example images that track finger movement from a clenched first position in FIG. 6C to an open hand position in FIG. 6D. The images of FIGS. 6E and 6F track motion from an open hand position in FIG. 6E to a pincer position of the index finger touching the thumb in FIG. 6F. As can be seen from FIGS. 6A-6F, the ability to track finger movements can even further enhance the remote motor assessments of the present disclosure, such as by evaluating movements at a more detailed level and customizing rehabilitation more specifically for the patient, compared to tracking only the overall limbs.

The Intel RealSense Skeleton Tracking SDK with Depth Mapping employs cameras that use active infrared stereo vision to provide accurate depth and position of objects within its field of view, while the SDKs use this data to build a skeleton model with 18 joints within the body frame in 2D/3D. This camera (or similar cameras) allows the measurement and comparison of specific joint angles and upper/lower extremity rotation when completing specific and active movements of the upper/lower extremities. Conventionally, these measurements and comparisons are completed by visual observation of the assessor. Implementation of skeleton tracking with depth mapping allows the addition of precision and accuracy to visual observation assessment by the clinician. Intel RealSense Hand Tracking allows the joints and bones of the hands to be tracked using 22 points of tracking. This tracking system (or other motion tracking software) can assess hand movements as directed when completing motor assessments such as the RAE-FM. The tablet stand is designed to position the cameras and the user interface—such as the 15″ IpsiHand touchscreen tablet personal computer (PC) and the depth cameras (e.g., Intel D435 or D455) and tracking cameras (e.g., Intel T265)—at a comfortable angle for seated assessment. The cameras may connect to the tablet PC via, for example, USB ports.

The systems and methods also include a unique real-time AI tracking model. The tracking framework is presented with a series of multiple raw joint locations that are continuously extracted from the sensor camera. The following description shall use 22 locations as utilized by the Intel RealSense Hand Tracking system; however, other numbers of joint locations may be used as appropriate for other tracking systems. Also, the description shall use the example of an upper extremity evaluation (FMA-UE) but may also apply to the lower extremity.

The motion of a subject is described by the relative position of the points over time. In the tracking model of the present disclosure, each point i is characterized by a state x_{t,i}∈R³ that defines the location of the joint at time t. To each state x_{t,i} is associated an observation y_{t,i}∈R³ that depicts the position of the joint measured on the current frame. Observations differ from the state x_{t,i} because they come from a joint detection algorithm that can be affected by noise and artifacts, whereas the value x_{t,i} is obtained through inference, thus believed to be more robust. Detecting joints in a frame at time t amounts to estimating p(xₜ|y{1 . . . t}), the posterior belief associated with the states xₜ={x_{t,1}, . . . , x_{t,22}} given all observations yₜ={y_{t,1}, . . . , y_{t,22}} accumulated so far. Using the Markov assumption and taking into account the dependence between the different joints p(x_{t,i}|x_{t,j}) and between successive time points p(x_{t,i}|x_{t-1,i}), the posterior marginal of the first joint can be written:

p(x_{t,1}|y_{{1 . . . t}})=p(y_{t,1}|x_{t,1})∫p(x_{t-1,1})p(x_{t-1,1}|y_{{1 . . . t-1}})dx_{t-1,1}

∫p(x_{t,1}|x_{{t,2}})p(x_{t,2}|y_{{1 . . . t}})dx_{t,2} . . . ∫p(x_{t,1}|x_{{t,22}})p(x_{t,22}|y_{{1 . . . t}})dx_{t,22}

In an example embodiment, a dynamic Markov model may be used to estimate the solution of this equation. The model describes relationships between pairs of nodes using the three types of functions. Observation potentials ϕ(x_{t,i},y_{t,i}) link observations y_{t,i} to their state x_{t,i} using a Gaussian model. Compatibility potentials ψ_{i,j}(x_{t,i},x_{t,j}) are represented by a kernel density estimation (KDE) that is constructed by collecting a set of joint positions across the training set. Finally, temporal potentials ψ(x_{t,i},x_{t-1,i}) define the relationship between two successive states of a joint using a kernel density estimation model. Joint localization is achieved through inference using Nonparametric Belief Propagation (NBP). After inference, the time-series are labelled based on the individual motions of the FMA-UE (or other motor assessment that is being performed). A Long Short-Term Memory Network (LSTM) is trained in a supervised manner to map the dynamics of each filtered joints to its corresponding label. LSTM is a variation of the recurrent neural network (RNN) which allows information to persist inside the network via a loopy architecture. LSTMs are particularly well suited to represent time series and are used in the framework to model the relationship between motion captured over time on the Intel RealSense and the motion label. The FMA-UE score, which has been assessed remotely, is then obtained by aggregating the output of the LSTM over a series of motions.

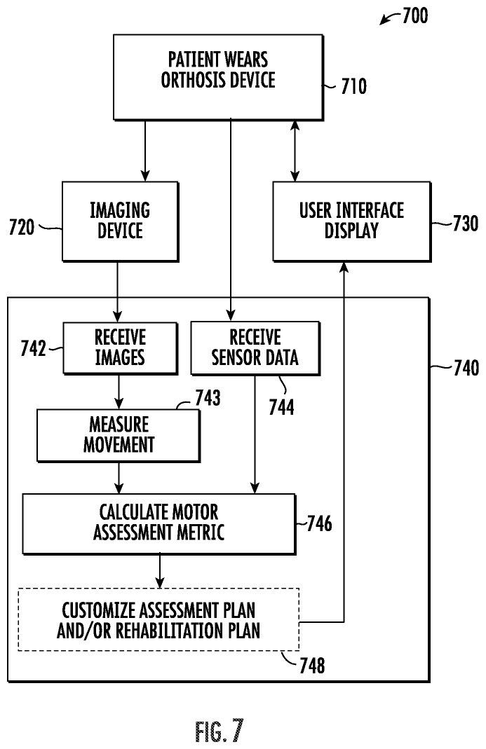

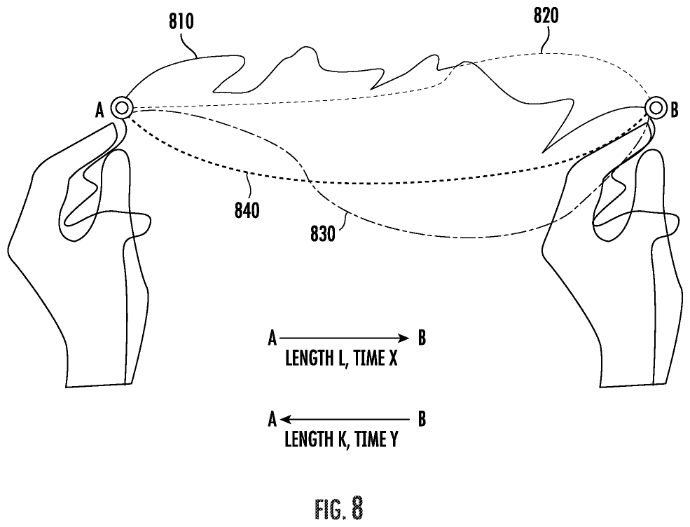

FIG. 7 is a block diagram 700 representing systems and methods for performing remote measurements in accordance with embodiments of the present disclosure. In block 710 a patient wears an orthosis device, such as the orthosis device 100 as described previously, or another orthosis device for the upper or lower extremity. The orthosis is a wearable device that can be utilized by a patient at home. The orthosis device has a sensor such as one or more of a force sensor (e.g., force sensing resistor or load cell as described previously), a position sensor, an accelerometer or a gyroscope. The patient receives instructions for performing a motor assessment task via a user interface display 730 such as a tablet computer (e.g., computing device 420 of FIG. 4). A custom graphic user interface (GUI), such as the user interface 428 shown on the tablet computing device 420 in FIG. 4, steps the patient through a test sequence and demonstrates the appropriate motion while the patient performs the test.

An imaging device 720 (e.g., depth camera 410, tracking camera 415 of FIG. 4) records images such as videos or a series of static images while the patient performs the motor assessment tasks. The images are received by a computer 740 that includes instructions that cause the computer to perform a method. The computer 740 may be the same device as the user interface display 730 and/or may include a separate computer processor (e.g., central processor 430 of FIG. 4). The method performed by computer 740 includes block 742 of receiving images from the imaging device and block 743 of making a measurement of a movement from the images. The movement is performed by the human patient. The measurement of the movement in block 743 may be made, for example, by skeleton tracking software as described above. Block 744 involves receiving sensor data from the orthosis device. Block 746 involves calculating a motor assessment metric using the measurement of the movement from block 743 and data from the sensor of the orthosis device from block 744.

An optional block 748 may include customizing a motor assessment plan and/or a rehabilitation plan for the patient. The method performed by computer 740 uses software algorithms that may include machine learning in some embodiments, and may personalize the instructions over time based on the patient's needs and progress. For example, the software algorithms may customize the motor assessment instructions by omitting or adding certain motions, or providing more or less detailed guidance for particular motions depending on how well the patient has performed the motions in the past. Time-varying 3D joint position data may be processed to determine movement rate, quality of motion and change in position, either absolutely or compared to the unaffected side. Joint position data and video images may be uploaded to a cloud server for offline review. The user interface can include the ability for remote patient management by a therapist using the built-in tablet PC camera to allow real time assistance to the patient.

The systems and methods of the present disclosure beneficially utilizes sensor data from the orthosis device along with motor movement measurements to derive assessment metrics. As an example, in addition to the motion evaluations assessed visually by skeletal tracking, some embodiments may also include force measurements to perform the grip strength (grasp) assessments of the FMA-UE or other tests. In a specific example, the orthosis device (e.g., Neurolutions IpsiHand) may be configured to have its force sensors measure flexion and extension against a prescribed resistance, and these forces may be used to derive the grasp evaluations. In further examples, force sensors in the orthosis device may be used to measure the flexion and extension forces of the patient's fingers when holding or pulling on a particular object, or when trying to resist a movement actuated by the orthosis device.

Other sensors that may be used in addition to those mentioned elsewhere in this disclosure include, but are not limited to, electrical current sensors, electromyography sensors, electrocardiograms, temperature sensors, and biometric sensors such as pulse/heart rate, oximeter, and stress/perspiration (e.g., analyte/molecular sensors for specific biological substances). Data from the various sensors (e.g., environmental sensors 440 and biological sensors 445 of FIG. 4) can be used, for example, to derive the amount of effort required by the patient for particular movements, or to determine environmental conditions (e.g., temperature) that may impact the patient's performance during an evaluation. Any of the sensors in this disclosure may be used alone or in combination with each other to assess information about the patient's rehabilitation progress, to customize therapy plans, and predict outcomes for the patient. In some embodiments, measurements from these sensors as well as interactions between the measurements (e.g., correlation, coherence) can be used as metrics in the remote assessments of the present disclosure. For example, cortico-muscular measurements may correlate with Fugl-Meyer scores.

In some embodiments, reflex items of the FMA-UE can be included in the remote assessment methodology of the present disclosure, such as by having the patient perform actions similar to conventional reflex assessments. In other embodiments, reflex evaluations may be omitted from the assessment based on the patient's needs.

Embodiments of the present disclosure enable home-based, personalized assessment and treatment of chronic stroke patients, allowing not only more patients to access rehabilitation services but also increasing the quality of those services through the customized analysis and monitoring provided by the AI algorithms and software. Patients can differ greatly from each other in their rehabilitation progress. For example, some patients may progress in a linear path of steady improvement in multiple areas. Other patients may have more circuitous progress, sometimes improving and sometimes regressing, with certain motions making more or less progress than others. With the present systems and methods, the RAE (e.g., RAE-FM) can assess the individual's status over time and adapt accordingly. For example, the algorithm may identify a particular joint or type of motion that is not progressing as well as others, and then personalize the RAE to take more measurements in those areas. In areas that have faster progress, the algorithm may tailor the assessment to conduct those measurements less frequently, or to perform some movements in combination with other movements to streamline the evaluation. In other words, the automated assessment can customize the evaluation for the needs of the individual patient and adapt over time as the patient proceeds along their rehabilitation.

FIG. 8 shows an example of how the present systems and methods can beneficially provide unique assessment aspects in evaluating a patient's progress and individually tailoring their therapy, all performed remotely. FIG. 8 shows a diagram of a patient's afflicted hand moving from point A to point B during a motor assessment. Points A and B may be, for example, the ipsilateral ear and the contralateral knee as illustrated in FIGS. 5A-5B, or any other end points required by an assessment (including movements by other upper extremity or lower extremity appendages). “L” is the path length from A to B, while “K” is the path length from B to A. L and K can be different from each other during an assessment due to the patient having more difficulty moving in one direction than another. The path lengths L and K may be determined by the skeletal tracking system described above, along with the speed of movement where “x” is the time to travel from A to B and “y” is the time to travel from B to A. In some embodiments, sensors on an orthosis device (e.g., device 100) can also be used to make measurements (e.g., position, speed, acceleration, orientation) in conjunction with the skeletal tracking system during the assessment. The path lengths L and K can be measured in 3D space and optimized via therapy to achieve shorter lengths over time, reflecting improved motor control. The time x and y to travel the distances L and K, respectively, can also be measured and optimally minimized via therapy to reflect improved motor control.

Path 810 represents an assessment movement during an acute stage of recovery. As can be seen, path 810 is very circuitous due to the patient lacking significant motor control during this early stage. Paths 820 and 830 represent assessment movements at intermediate stages of rehabilitation, the paths 820 and 830 being shorter and smoother between A and B but still not optimized. Path 840 represents an assessment movement at a later stage of rehabilitation, being more direct and smoother than paths 810, 820 or 830—thus showing improved motor control.

In embodiments of FIG. 8, L, K, x and y are individually optimized toward minimum values tailored for that specific patient to achieve a positive impact on motor therapy. L and K will approach each other asymptotically with improved motor control, as will x and y. Measurements of L and K, as well as x and y, on the unafflicted side may be used as baseline measures for representation of optimum length and time, respectively. Hand dominance may impact what values are chosen for these baseline measures. For example, if the afflicted side is the non-dominant hand, a goal may be set to achieve 80% of the lengths and times (L and K, x and y) performed by the unafflicted, dominant hand. Analysis of further details of the motions can also be performed with the skeletal tracking system, such as identifying that a patient is having more difficulty with movement near the end of the movement range than at the beginning (e.g., as indicated by slower speed and/or more circuitous route). In such a case, therapy can be prescribed for that individual patient to improve movement of that specific range of motion. As demonstrated by FIG. 8, the present methods and systems enable more detailed, quantitative assessments than conventional methods which tend to be qualitative (e.g., 0—none, 1—partial, 2—full for Fugl-Meyer). Furthermore, the present systems enable human motor assessments to be performed remotely, in a location separate from the patient (e.g., patient at their home, medical professional at an office), rather than requiring the in-person presence of a medical professional.

In embodiments, systems for performing human motor assessments remotely (i.e., a patient and medical professional in different locations from each other) include a wearable orthosis device having a sensor, an imaging device, and a computer. The computer, such as the computing device 420 and/or central processor 430 of FIG. 4, includes instructions that cause the computer to perform a method. The method includes receiving images from the imaging device; making a measurement of a movement from the images, the movement performed by the patient; and calculating a motor assessment metric using the measurement of the movement and data from the sensor. The system may further include a brain-computer interface in communication with the wearable orthosis device.

The sensor may be, for example, a force sensor (e.g., a force sensing resistor or a load cell), a position sensor, an accelerometer, or a gyroscope. Input from the sensors can be used with the skeleton tracking measurements to derive more accurate and/or additional metrics than what can be analyzed by the skeleton tracking measurements alone. For example, coordination or speed tests can be quantified using a position sensor and/or accelerometer, rather than scoring on a qualitative basis (e.g., the conventional FMA scale is ≥6 sec, 2-5 sec, <2 sec). In another example, a gyroscope can be used to quantify the amount of tremor during the movement, rather than the qualitative assessment of marked/slight/none of the conventional FMA. In further examples, the system can assess how well a patient performs volitional movements, such as using an accelerometer to detect if the patient slows down (i.e., has more trouble) at the end of the movement, or using a gyroscope to see how they steady the patient's movements are.

In some embodiments, the method performed by the computer further comprises recording an environmental input or a biometric input when making the measurement of the movement. As an example, the temperature of the room can be used for correlating how the environment affects the patient's performance. In another example, the amount of effort required by the patient for making certain movements can be assessed by measurements of heartrate or oxygen level from a pulse oximeter. This information from environmental or biometric sensors can enhance the understanding of the patient's progress and enable the system to make recommendations better suited for the individual patient. For example, the system can recognize that the ambient environment may have detrimentally affected the patient's performance that day. In another example, the system can use heartrate information to note that a movement in one direction is more difficult than in the opposite direction, even though both movements may have been performed at the same speed or accuracy.

In some embodiments, the method performed by the computer further comprises customizing a therapy plan for the patient based on the motor assessment metric and the measurement. The computer can analyze measurements from the remote motor assessments over time and revise the assessment routine accordingly. For example, if a patient is making good progress in one type of motion, the system can recommend certain tests that are aimed at that motion to be conducted less frequently or to be omitted. In another example, if the patient is having trouble in one type of motion, the system can focus the remote assessment around tests that target that motion. The system can also provide metrics to the patient that quantify amounts of progress, such as from data provided by sensors on the orthosis device, to provide motivation for the patient. The customized therapy plan can streamline testing routines, target trouble areas more specifically, and improve patient compliance by motivating the patient through metrics on their progress. The patient's physician or physical therapist can also view results and data from the motor assessment system and make changes to the rehabilitation plan or ongoing assessment test plans accordingly.

In some embodiments, the wearable orthosis device comprises a body part interface and a motor-actuated assembly coupled to the body part interface. The body part interface may be attachable to a finger (any finger including the thumb), hand, wrist, forearm, shoulder, toe, foot, ankle, shin, thigh, or other body part. The motor-actuated assembly can beneficially assist the patient in performing movements, while also gathering information for the remote assessment such as an amount of assistance being provided for the movement. The movement may be, for example, hand movement, finger movement, or movement of other parts of the body such as the arm, shoulder, foot, or leg. In some embodiments, the sensor is coupled to the body part interface. For example, the body part interface may be attachable to a finger, and the data from the sensor may be a gripping force, or a force exerted by the patient for extending or flexing the finger. In some embodiments, the motor-actuated assembly is configured to assist the movement performed by the patient.

As has been described herein, the systems and methods of the present disclosure combine a wearable orthosis device with an imaging device and customized software to beneficially enable human motor assessments to be performed remotely. The systems and methods provide unique features such as new metrics and quantifiable measurements compared to what can be performed with conventional motor assessments.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A system for performing human motor assessments remotely, comprising:
a wearable orthosis device having a sensor;
an imaging device; and
a computer including instructions that cause the computer to perform a method, the method comprising:
receiving images from the imaging device;
making a measurement of a movement from the images, the movement performed by a patient; and
calculating a motor assessment metric using the measurement of the movement and data from the sensor.

2. The system of claim 1, wherein the sensor is a force sensor.

3. The system of claim 1, wherein the sensor is a position sensor, an accelerometer or a gyroscope.

4. The system of claim 1, wherein the method performed by the computer further comprises recording an environmental input or a biometric input when making the measurement of the movement.

5. The system of claim 1, wherein the method performed by the computer further comprises customizing a therapy plan for the patient based on the motor assessment metric and the measurement of the movement.

6. The system of claim 1, wherein the wearable orthosis device comprises a body part interface and a motor-actuated assembly coupled to the body part interface.

7. The system of claim 6, wherein the body part interface is attachable to a finger.

8. The system of claim 1, wherein the movement is hand movement or finger movement.

9. The system of claim 1, wherein the motor assessment metric is a metric in a Fugl-Meyer assessment, a motricity index, an action research arm test (ARAT), an arm motor ability test (AMAT), or a stroke impact scale (SIS).

10. The system of claim 1, wherein the system is configured to communicate with a mobile device that display directions for performing the movement.

11. The system of claim 1, further comprising a brain-computer interface in communication with the wearable orthosis device.

12. A system for performing human motor assessments remotely, comprising:
a wearable orthosis device having a body part interface, a motor-actuated assembly coupled to the body part interface, and a sensor coupled to the body part interface;
an imaging device; and
a computer including instructions that cause the computer to perform a method, the method comprising:
receiving images from the imaging device;
making a measurement of a movement from the images, the movement performed by a patient; and
calculating a motor assessment metric using the measurement of the movement and data from the sensor.

13. The system of claim 12, wherein the sensor is a force sensor.

14. The system of claim 13, wherein:
the body part interface is attachable to a finger; and
the data from the sensor is a gripping force, or a force exerted by the patient for extending or flexing the finger.

15. The system of claim 12, wherein the motor-actuated assembly is configured to assist the movement performed by the patient.

16. The system of claim 12, wherein the sensor is a position sensor, an accelerometer or a gyroscope.

17. The system of claim 12, wherein the method performed by the computer further comprises recording an environmental input or a biometric input when making the measurement of the movement.

18. The system of claim 12, wherein the method performed by the computer further comprises customizing a therapy plan for the patient based on the motor assessment metric and the measurement of the movement.

19. The system of claim 12, wherein the motor assessment metric is a metric in a Fugl-Meyer assessment, a motricity index, an action research arm test (ARAT), an arm motor ability test (AMAT), or a stroke impact scale (SIS).

20. The system of claim 12, further comprising a brain-computer interface in communication with the wearable orthosis device.
